(19) United States
(12) Patent Application Publication
(10) Pub. No.: US 2018/0360769 A1
(43) Pub. Date: Dec. 20, 2018

(54) SYSTEMS AND METHODS FOR TRANSDERMAL DRUG DELIVERY

(71) Applicant: Elliptical Therapeutics, LLC, San Diego, CA (US)

(72) Inventor: Samir ROY, San Diego, CA (US)

(21) Appl. No.: 15/779,940

(22) PCT Filed: Nov. 28, 2016
(86) PCT No.: PCT/US16/63816

§ 371 Date: May 30, 2018

Related U.S. Application Data
(60) Provisional application No. 62/260,980, filed on Nov. 30, 2015.

(51) Int. Cl. A61K 9/70; A61K 9/00; A61K 31/407; A61K 47/10; A61K 47/12; A61K 47/38; A61K 47/32; A61K 47/14

(52) U.S. Cl. CPC A61K 9/7084; A61K 9/0014; A61K 31/407; A61K 47/14; A61K 47/12; A61K 47/38; A61K 47/32; A61K 47/10

(57) ABSTRACT

A liquid-reservoir system (LRS) for transdermal administration of (s)-ketorolac may include a backing layer that is substantially impermeable to (s)-ketorolac, isopropyl alcohol, isopropyl myristate, oleic acid, butylated hydroxytoluene, triethanolamine, hydroxypropyl cellulose, and water; it may additionally include an (s)-ketorolac reservoir layer including a liquid-reservoir gel formulation, a membrane layer, and a contact adhesive layer. The liquid-reservoir gel formulation may include: 1-15% by weight (s)-ketorolac or a salt thereof; one or more drug solubilizing vehicles; one or more permeation enhancers; an antioxidant; a thickening agent; purified water; and a buffering agent. A method for transdermal drug delivery includes: providing an LRS; removing a release liner layer by peeling away the release liner layer, positioning the LRS onto a patient's skin with a backing layer facing outward from the patient's skin; and applying pressure to the backing layer to non-permanently adhere the LRS to the patient's skin.

### CROSS-REFERENCE TO RELATED APPLICATIONS

This application claims priority to U.S. provisional patent application Ser. No. 62/260,980, filed Nov. 30, 2015, titled “SYSTEMS AND METHODS FOR TRANSDERMAL DRUG DELIVERY,” the disclosure of which is herein incorporated by reference in its entirety.

### TECHNICAL FIELD

This invention relates generally to the field of drug delivery, and more specifically to new and useful systems and methods for transdermal drug delivery.

### BACKGROUND

Ketorolac tromethamine (Toradol®) is a non-narcotic and nonsteroidal agent with potent analgesic and moderate anti-inflammatory activities. Clinical studies with Toradol® have demonstrated that a single-dose of Toradol® is more efficacious than that of morphine, meperidine, or pentazocine in managing moderate to severe post-operative pain. Toradol® does not pose potential addiction problems or respiratory depression, both of which are adverse effects commonly associated with narcotic analgesics therapy. Typically, Toradol® is administered by intramuscular injection, perorally, or intranasally. When Toradol® is administered orally for extended periods of time, gastric irritation and ulcers have been observed; such adverse effects have limited the duration of use of Toradol®.

Ketorolac is a chiral drug and exists as a racemic mixture of s and r enantiomers. It is reported that essentially all the pharmacological activity resides in the s-enantiomer, which is approximately twice as potent as the (r)-enantiomer in animal models. In human subjects, a single oral solution dose of racemic ketorolac (30 mg), (s)-ketorolac (15 mg), or (r)-ketorolac (15 mg) resulted in plasma concentrations of (s)-ketorolac that were lower, after all sample times, than plasma concentrations of (r)-ketorolac were after oral administration of racemic ketorolac. In comparing racemic ketorolac, (s)-ketorolac, and (r)-ketorolac, plasma half-life was shorter and clearance greater for (s)-ketorolac than for (r)-ketorolac. Because the biological half-life of racemic ketorolac is reported to be 4-6 hours, frequent dosing is necessary to maintain therapeutic effect of the drug to alleviate pain in post-operative patients. Currently, racemic ketorolac tromethamine (Toradol®) is administered orally four times daily for five days.

Thus, there is a need for new and useful systems and methods for delivery of ketorolac, which will reduce the dosing frequency required by oral administration by maintaining steady-state plasma levels over 24 hours and lower the systemic adverse events often seen following oral administration. This disclosure provides such a new and useful systems and methods.

### SUMMARY

The present disclosure is directed to improved treatment of post-operative pain with reduced systemic side-effects. One aspect of the disclosure is directed to transdermal delivery of (s)-ketorolac for the treatment of post-operative pain. Another aspect of the disclosure is directed to a liquid-reservoir system for transdermal delivery of (s)-ketorolac to limit the amount of (s)-ketorolac required to reduce post-operative pain.

One aspect of the disclosure is directed to a liquid-reservoir system for transdermal administration of (s)-ketorolac. In various embodiments, the liquid-reservoir system includes a backing layer that is substantially impermeable to (s)-ketorolac, isopropyl alcohol, isopropyl myristate, oleic acid, butylated hydroxytoluene, triethanolamine, hydroxypropyl cellulose, and water. The liquid-reservoir system of various embodiments further includes an (s)-ketorolac reservoir layer comprising a liquid-reservoir formulation. In some embodiments, the backing layer defines a front surface or face of the liquid-reservoir system. In some embodiments, the liquid-reservoir formulation includes: 1-15% by weight (s)-ketorolac or a salt thereof, one or more drug solubilizing vehicles, one or more permeation enhancers, an antioxidant, a thickening agent, purified water, and a buffering agent. In some embodiments, the liquid-reservoir formulation is a liquid gel.

In some embodiments, the liquid-reservoir system exhibits in vitro skin flux through a skin surface of a cadaver in a range of about 5 to about 100 μg/cm²/hr. In some embodiments, the liquid-reservoir system administers at least about 25% of the (s)-ketorolac during approximately 24 hours of use.

In some embodiments, a pH of the reservoir layer ranges from 3.5 to 6.5. In some embodiments, the backing layer is occlusive and heat-sealable to a membrane layer. In some embodiments, a basal surface area of the liquid reservoir system is 10 to 100 cm².

In some embodiments, the (s)-ketorolac forms 2% to 5% of the reservoir layer by weight. In some embodiments, the purified water forms 20% to 60% of the reservoir layer by weight. In some embodiments, the purified water forms 25% to 50% of the reservoir layer by weight.

In some embodiments, the one or more drug solubilizing vehicles include one or more of: isopropyl alcohol or ethanol. In some embodiments, one or more of the isopropyl alcohol or ethyl alcohol form 25% to 75% of the reservoir layer by weight. In some embodiments, one or more of the isopropyl alcohol or ethyl alcohol form 35% to 65% of the reservoir layer by weight.

In some embodiments, the one or more permeation enhancers include one or more of isopropyl myristate, oleic acid, and propylene glycol monolaurate. In some embodiments, one or more of the isopropyl myristate, oleic acid, and propylene glycol monolaurate forms 0.5% to 10% of the reservoir layer by weight. In some embodiments, one or more of the isopropyl myristate, oleic acid, and propylene glycol monolaurate forms 1.5% to 4% of the reservoir layer by weight.

In some embodiments, the antioxidant includes one or more of tocopherol and butylated hydroxytoluene. In some embodiments, one or more of the tocopherol and butylated hydroxytoluene form 0.5% to 3% of the reservoir layer by weight. In some embodiments, one or more of the tocopherol and butylated hydroxytoluene form 0.1% to 0.3% of the reservoir layer by weight.

In some embodiments, the thickening agent includes one or more of hydroxypropyl cellulose, hydroxyethyl cellulose, and carboxymethyl cellulose. In some embodiments, one or more of the hydroxypropyl cellulose, hydroxyethyl cellulose, and carboxymethyl cellulose form 0.5% to 5% of the reservoir layer by weight. In some embodiments, one or more of the hydroxypropyl cellulose, hydroxyethyl cellulose, and carboxymethyl cellulose form 1% to 3% of the reservoir layer by weight.

In some embodiments, the system further includes a membrane layer, which is formed, at least in part, by a microporous polyethylene membrane or an ethylene-vinyl-acetate membrane. In some embodiments, the membrane layer includes an ethylene-vinyl-acetate membrane having an amount of vinyl acetate between 9% and 28%.

In some embodiments, a thickness of the membrane layer is 0.02 mm, 0.2 mm, or any value there between. In some embodiments, a thickness of the membrane layer is 0.05 mm, 0.15 mm, or any value there between.

In some embodiments, the system further includes a contact adhesive layer formed, at least in part, of a pressure-sensitive adhesive and a tackifier. In some embodiments, the contact adhesive layer is formed of: one or more of an acrylates copolymer, silicone, or polyisobutylene pressure-sensitive adhesive; and one or more of a mineral oil or silicone oil tackifier. In some embodiments, one or more of the mineral oil or silicone oil form 1% to 5% of the contact adhesive layer by weight. In some embodiments, one or more of the mineral oil or silicone oil form 0.5% to 2% of the contact adhesive layer by weight.

Another aspect of the disclosure is directed to a method of transdermal drug delivery. In various embodiments, the method includes providing a liquid-reservoir system. The liquid-reservoir system of various embodiments includes a backing layer, a liquid-reservoir layer, a membrane layer, a contact adhesive layer, and a release liner layer. The method of various embodiments further includes: removing the release liner layer from the liquid-reservoir system by peeling away the release liner layer; positioning the liquid-reservoir system onto a portion of a patient's skin with the backing layer facing outward from the portion of the patient's skin; and applying pressure to the backing layer to non-permanently adhere the liquid-reservoir system to the portion of the patient's skin. In some embodiments, the liquid-reservoir layer includes an active drug ingredient, one or more permeation enhancers, purified water, an antioxidant, and a buffering agent. In some embodiments, the active drug ingredient is (s)-ketorolac or a salt thereof.

### BRIEF DESCRIPTION OF THE DRAWINGS

FIG. 1A is a schematic side view of one embodiment of an (s)-ketorolac liquid reservoir system.

FIG. 18 is a top view of one embodiment of an (s)-ketorolac liquid reservoir system.

FIG. 2 is a flow chart of a method of transdermal drug delivery.

FIG. 3 shows a Franz cell diffusion system to assess (s)-Ketorolac gel formulation skin permeation.

FIG. 4 shows a histogram depicting a steady-state skin flux (μg/cm²/h) of(s)-Ketorolac gel formulation (KRG-1) or liquid-reservoir system formulations (LRS-1, LRS-2, LRS-3) that permeated cadaver skin in the Franz cell diffusion system of FIG. 3.

FIG. 5 shows a line graph depicting a percent label strength over time (months) of (s)-Ketorolac gel formulation (KRG-5) stored at 25° C. and 40° C.

### DETAILED DESCRIPTION

The foregoing is a summary, and thus, necessarily limited in detail. The above-mentioned aspects, as well as other aspects, features, and advantages of the present technology will now be described in connection with various embodiments. The inclusion of the following embodiments is not intended to limit the invention to these embodiments, but rather to enable any person skilled in the art to make and use this invention. Other embodiments may be utilized and modifications may be made without departing from the spirit or the scope of the subject matter presented herein. Aspects of the disclosure, as described and illustrated herein, can be arranged, combined, modified, and designed in a variety of different formulations, all of which are explicitly contemplated and form part of this disclosure.

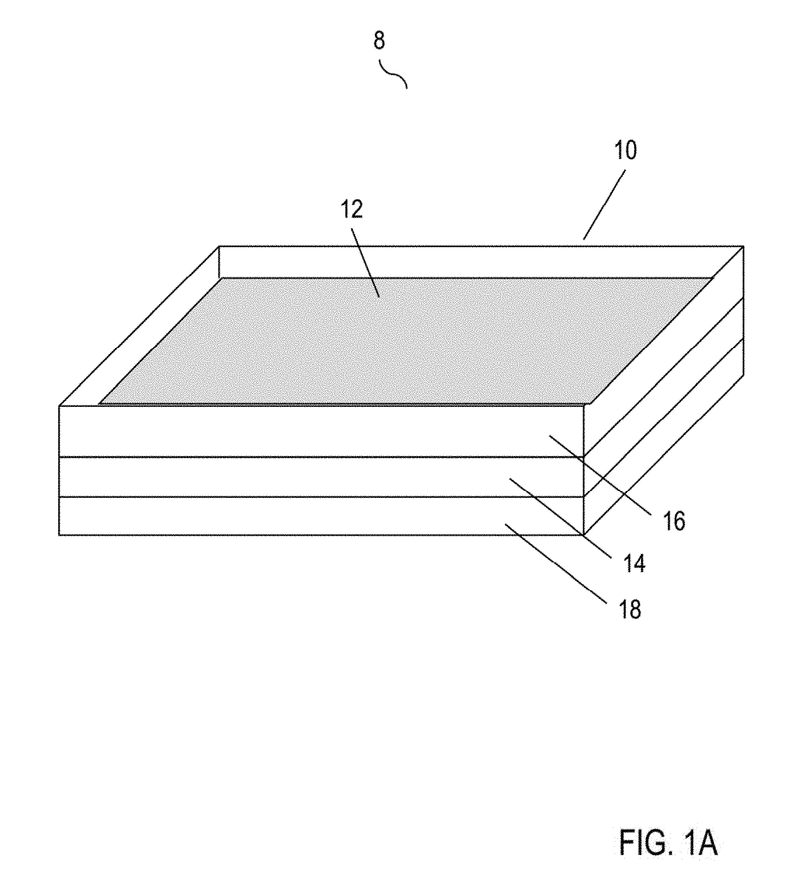

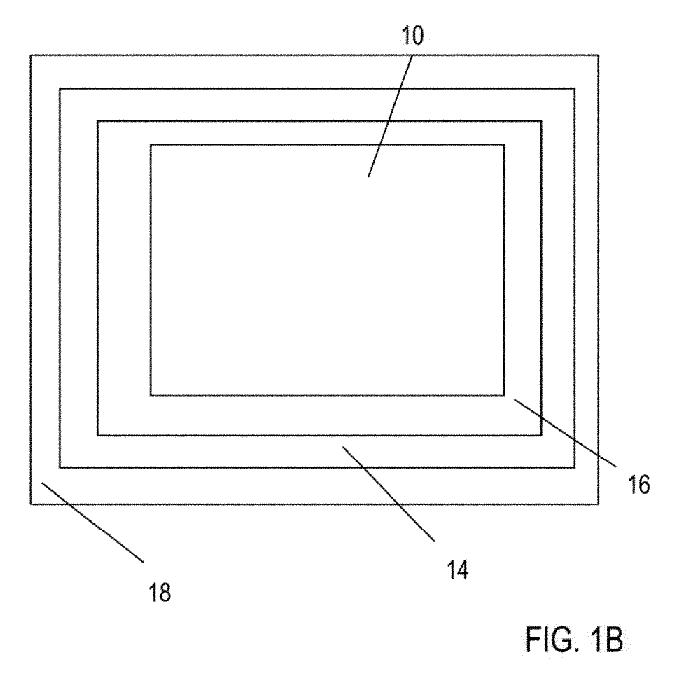

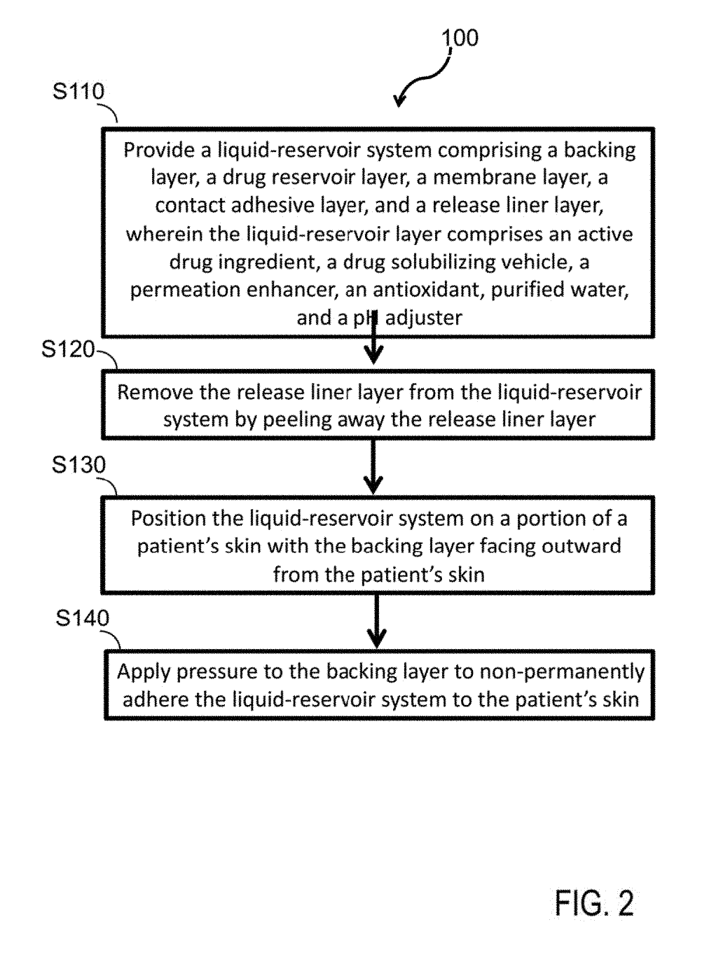

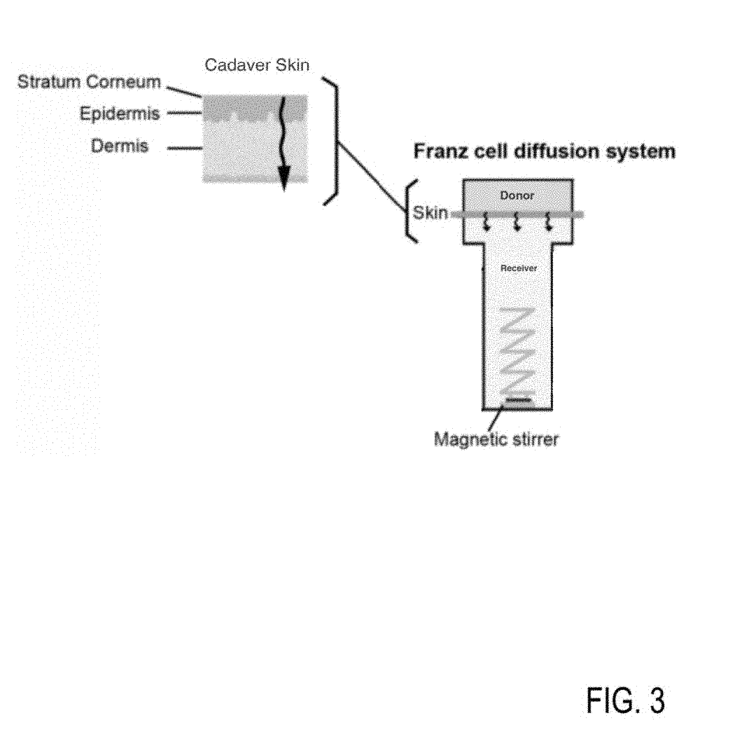

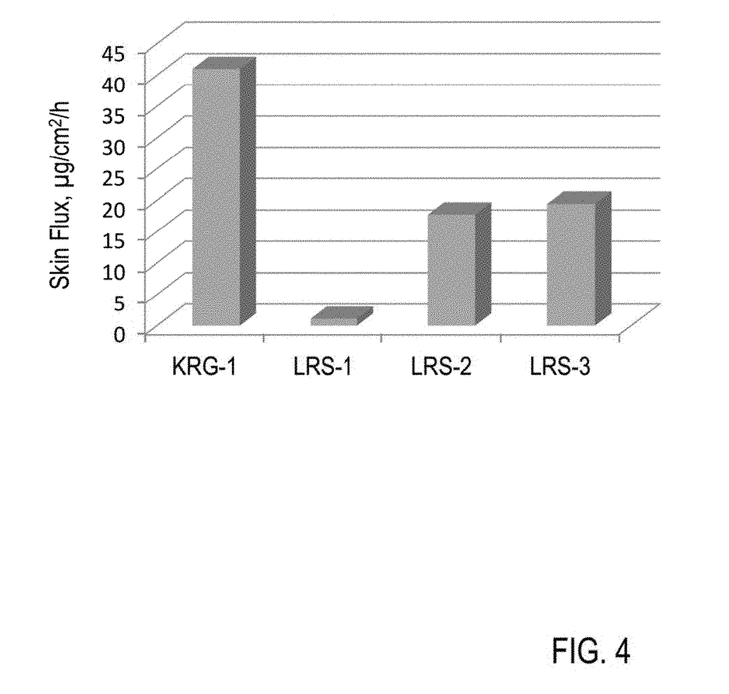

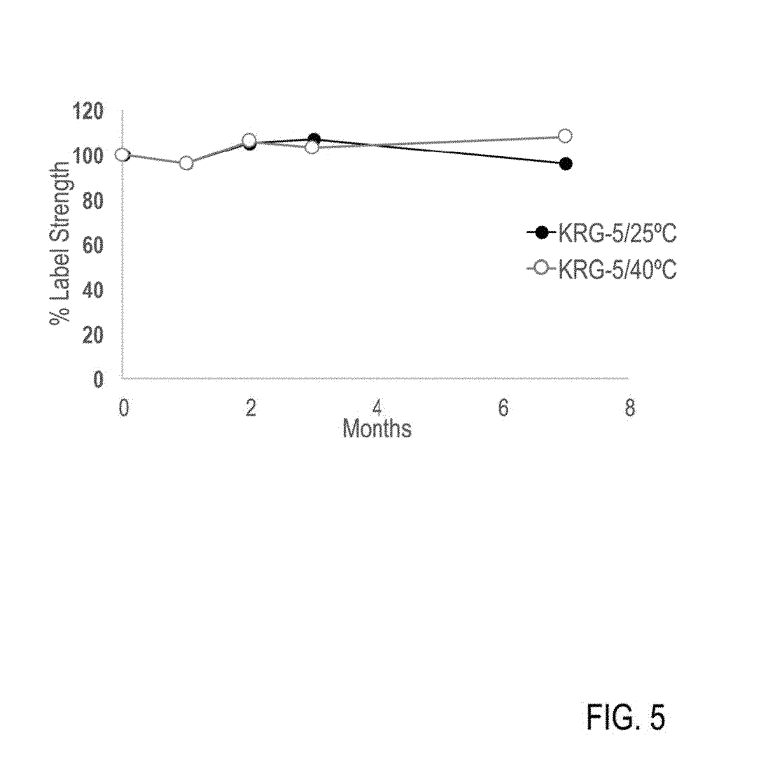

FIGS. 1A-1B illustrate a cross-sectional view and top view, respectively, of various embodiment of a liquid-reservoir system 8. In some embodiments, the liquid reservoir system functions to administer (s)-ketorolac transdermally. Alternatively or additionally, in some embodiments, the liquid reservoir system functions to administer a liquid analgesic, pain reliever, or other compound or composition. In some embodiments, the system includes five layers, including: a backing layer 10 that forms the upper (i.e., outermost) layer of the system, a reservoir layer 12, a membrane layer 16, a continuous contact adhesive layer 14 that also acts as a pressure sensitive adhesive (as shown in FIG. 1B), and a release liner layer 18. In other embodiments, the system includes a contact adhesive layer 14 positioned around a bottom perimeter or peripheral region or area of the liquid reservoir system. It shall be appreciated by one of skill in the art that the contact adhesive layer may take any shape or any structure or be continuous or discontinuous without departing from the original scope and intent of this disclosure.

In some embodiments, a basal surface area of the liquid-reservoir system is 10 to 100 cm². In some embodiments, a basal surface area of the liquid-reservoir system is 25 to 100 cm². In some embodiments, a basal surface area of the liquid-reservoir system is 50 to 100 cm². In some embodiments, a basal surface area of the liquid-reservoir system is 75 to 100 cm². In some embodiments, a basal surface area of the liquid-reservoir system is 10 to 50 cm².

In some embodiments, the liquid reservoir system is shaped similar to a rectangle, square, circle, hexagon, or any other shape. It shall be appreciated by one of skill in the relevant art that the liquid reservoir system may take any shape without departing from the original intent of this disclosure.

In some embodiments, the backing layer 10 is a structural layer that provides the liquid-reservoir system 8 with firmness. In some such embodiments, the backing layer 10 defines the front face (i.e., outer surface) of the liquid reservoir system, which when worn, is the layer positioned furthest from a patient's skin. The backing layer 10 is occlusive such that it is impermeable to (s)-ketorolac and inactive ingredients that may be present in the reservoir layer 12. In some such embodiments, the backing layer 10 may be made of an occlusive (i.e., water impermeable) material, for example, one or more of polyester, polyethylene, polypropylene, or any other occlusive material provided elsewhere herein or known to those skilled in the art. Further, in such embodiments, the reservoir layer 12 is placed and heat-sealed, for example between the backing layer 10 and the membrane layer 16. In some embodiments, a thickness of the backing layer 10 may be 25 μm-200 μm, 25 μm-100 μm, 100 μm-200 μm, or any thickness, thickness range, or thickness subrange therebetween. In some embodiments, a thickness of the backing layer 10 is 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 105 μm, 110 μm, 115 μm, 120 μm, 125 μm, 130 μm, 135 μm, 140 μm, 145 μm, 150 μm, 155 μm, 160 μm, 165 μm, 170 μm, 175 μm, 180 μm, 185 μm, 190 μm, 195 μm, 200 μm, or any suitable thickness.

In some embodiments, the reservoir layer 12 provides the liquid-reservoir for the drug, for example (s)-ketorolac. In some such embodiments, the reservoir layer 12 includes a gel (a homogeneous phase) including, for example, one or more of a drug; isopropyl alcohol; isopropyl myristate; oleic acid; propylene glycol monolaurate; purified water, tocopherol or butylated hydroxyl toluene (BHT); triethanolamine, tromethamine, dimethylamine, or epolamine; and hydroxypropyl cellulose (HPC) or hydroxyethyl cellulose.

In some embodiments, the reservoir layer 12 includes or is formed of a liquid gel. In some embodiments, the liquid gel includes weight by weight (w/w): 1-15% (s)-ketorolac; 30-80% isopropyl alcohol or ethyl alcohol; 0.5-5% isopropyl myristate; 0-5% oleic acid; 0.05-2% butylated hydroxytoluene (BHT); 0.5-5% triethanolamine, epolamine, and/or diethylamine as a buffering agent or pH adjuster; 20-60% purified water, and 1%-5% hydroxypropyl cellulose, hydroxyethyl cellulose or carboxymethyl cellulose. In some embodiments, the liquid gel has a pH between 3.5 and 6.5 or between 4.5 and 5.5.

In some embodiments, an (s)-ketorolac gel formulation includes 1-2%, 2-3%, 3-4%, 4-5%, 5-6%, 6-7%, 7-8%, 8-9%, 9-10%, 10-11%, 11-12%, 12-13%, 13-14%, or 14-15% (s)-ketorolac.

In some embodiments, an (s)-ketorolac gel formulation includes 30-40%, 40-50%, 50-60%, 60-70%, or 70-80% isopropyl alcohol or ethyl alcohol.

In some embodiments, an (s)-ketorolac gel formulation includes 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2-3%, 3-4%, 4-5% or any subrange therebetween of isopropyl myristate.

In some embodiments, an (s)-ketorolac gel formulation includes 0.0%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2-3%, 3-4%, 4-5%, or any subrange there between of oleic acid.

In some embodiments, an (s)-ketorolac gel formulation includes 0.05%, 0.06%, 0.07%, 0.08%, 0.09%, 0.1%, 0.11%, 0.12%, 0.13%, 0.14%, 0.15%, 0.16%, 0.17%, 0.18%, 0.19%, or 2% butylated hydroxytoluene.

In some embodiments, an (s)-ketorolac gel formulation includes 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2-3%, 3-4%, 4-5%, or any subrange there between of triethanolamine, epolamine, and/or diethylamine.

In some embodiments, an (s)-ketorolac gel formulation includes 1%, 2%, 1-2%, 2-3%, 3-4%, or 4-5% hydroxypropyl cellulose, hydroxyethyl cellulose, and/or carboxymethyl cellulose.

In one embodiment, an (s)-ketorolac gel formulation includes w/w: 3.2% (s)-ketorolac acid; 46.9% isopropyl alcohol; 1.1% isopropyl myristate; 0.07% butylated hydroxytoluene; 1.1% triethanolamine; 45.6% purified water; and 2% hydroxypropyl cellulose at a pH of 4.9.

In one embodiment, an (s)-ketorolac gel formulation includes w/w: 2.9% (s)-ketorolac acid; 53.9% isopropyl alcohol; 1.5% isopropyl myristate; 0.1% butylated hydroxytoluene; 1.5% epolamine; 38.1% purified water, and 2% hydroxypropyl cellulose at a pH of 5.6.

In one embodiment, an (s)-ketorolac gel formulation includes w/w: 2.9% (s)-ketorolac acid; 54.1% isopropyl alcohol; 1.7% isopropyl myristate; 2% oleic acid; 0.1% butylated hydroxytoluene; 1.4% triethanolamine; 35.9% purified water; and 2% hydroxypropyl cellulose at a pH of 4.9.

In one embodiment, an (s)-ketorolac gel formulation includes w/w: 3% (s)-ketorolac acid; 56.6% isopropyl alcohol; 2% isopropyl myristate; 0.1% butylated hydroxytoluene; 1.1% triethanolamine; 35.9% purified water; and 1.5% hydroxypropyl cellulose at a pH of 4.9.

In one embodiment, an (s)-ketorolac gel formulation includes w/w: 2.9% (s)-ketorolac acid; 46.8% isopropyl alcohol; 1.2% isopropyl myristate; 0.06% butylated hydroxytoluene; 1.1% triethanolamine; 45.9% purified water; and 2% hydroxypropyl cellulose at a pH of 4.9.

In some embodiments, the membrane layer 16 includes a microporous polypropylene membrane, ethylene vinyl acetate (EVA) membrane, or ultra high molecular weight polyethylene (e.g., Solupor-10P05A). In some such embodiments, the EVA membrane includes 1-30% vinyl acetate; in one embodiment, the EVA membrane includes 4-28% vinyl acetate. In some embodiments, the EVA membrane includes 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% vinyl acetate. Further, in some embodiments, the thickness of the membrane layer 16 ranges from 0.02-0.2 mm; in one embodiment, the thickness of the membrane layer 16 ranges from 0.02-0.1 mm or 0.1-0.2 mm. In some embodiments, the thickness of the membrane layer 16 is 0.02 mm, 0.04 mm, 0.06 mm, 0.08 mm, 0.1 mm, 0.2 mm, 0.4 mm, 0.6 mm, 0.8 mm, 1 mm, 1.1 mm, 1.2 mm, 1.4 mm, 1.6 mm, 1.8 mm, or 2 mm. In some embodiments, the membrane layer 16 is highly porous. In some such embodiments, the membrane layer 16 is over 50% porous, over 60% porous, over 70% porous, over 80% porous, over 90% porous, or over 95% porous.

In some embodiments, the contact adhesive layer 14 is configured for contacting and attaching to a skin surface (e.g., arm, leg, hand, stomach, chest, back, etc.) of a user. In some such embodiments, the contact adhesive layer 14 includes: a pressure-sensitive adhesive, for example, acrylates (e.g., Duro-Tak 2516, Duro-Tak 900A, Duro-Tak 9301, Duro-Tak 4098), silicones, polyisobutylene, or any combination thereof, and/or a tackifier, for example, mineral oil or silicone oil.

In some embodiments, prior to application of the liquid-reservoir system 8 to a skin surface of a patient, the liquid-reservoir system 8 includes a release liner layer 18 that covers a basal, bottom, or base surface of the contact adhesive layer 14. In some such embodiments, the release liner layer 18 functions to protect and/or cover the contact adhesive layer 14 until use. In some embodiments, the release liner layer 18 is removed from the contact adhesive layer 14 to expose the basal surface and allow the liquid-reservoir system 8 to be adhered to the skin. In some such embodiments, the release liner layer 18 is peelable or otherwise removable from the liquid-reservoir system 8 prior to use to expose the contact adhesive layer 14. The release liner layer 18 includes a polymer, for example, a polyester, that is substantially impermeable to one or more of: a drug, for example, (s)-ketorolac; isopropyl alcohol; isopropyl myristate; oleic acid; propylene glycol monolaurate; water; tocopherol; BHT; triethanolamine; tromethamine; dimethylamine; epolamine; hydroxypropyl cellulose (HPC); mineral oil; and/or silicone oil.

In some embodiments, an (s)-ketorolac composition includes one or more inactive ingredients. In some such embodiments, the inactive ingredients may include one or more of a permeation enhancer, an antioxidant, a thickening agent, purified water, and a buffering agent.

In some such embodiments, the drug solubilizing vehicle is isopropyl alcohol or ethanol, or any combination thereof.

In some such embodiments, the permeation enhancer is isopropyl myristate, oleic acid, propylene glycol monolaurate, or any combination thereof. In other embodiments, any other suitable permeation enhancer or combination of permeation enhancers may be used.

In some embodiments, the antioxidant is tocopherol, butylated hydroxytoluene, or a combination thereof. In other embodiments, any other suitable antioxidant or combination of antioxidants may be used.

In some embodiments, the thickening agent is hydroxypropyl cellulose, hydroxyethyl cellulose, carboxymethyl cellulose, or a combination thereof. In other embodiments, any other suitable thickening agent or combination of thickening agents may be used.

In some embodiments, the buffering agent is triethanolamine, epolamine, diethylamine, tromethamine, or a combination thereof. In other embodiments, any other suitable buffering agent or combination of buffering agents may be used.

In some embodiments, the liquid-reservoir system 8 exhibits a steady-state (s)-ketorolac skin flux in vitro in the range of about 5 to about 100 g/cm²/hr. In some embodiments, the liquid-reservoir system 8 exhibits a steady-state (s)-ketorolac skin flux in vitro in the range of about 5 to about 50 μg/cm²/hr. In some embodiments, the liquid-reservoir system 8 exhibits a steady-state (s)-ketorolac skin flux in vitro in the range of about 50 to about 100 μg/cm²/hr. In some embodiments, the liquid-reservoir system 8 exhibits a steady-state (s)-ketorolac skin flux in vitro in the range of about 25 to about 100 μg/cm²/hr. In some embodiments, the liquid-reservoir system 8 exhibits a steady-state (s)-ketorolac skin flux in vitro in the range of about 5 to about 75 μg/cm²/hr.

Further, in some embodiments, the liquid-reservoir system 8 administers at least about 25% of the (s)-ketorolac in the reservoir system during approximately 24 hours of use. Further, in some embodiments, the liquid-reservoir system 8 administers at least about 10% of the (s)-ketorolac in the reservoir system during approximately 24 hours of use. Further, in some embodiments, the liquid-reservoir system 8 administers at least about 15% of the (s)-ketorolac in the reservoir system during approximately 24 hours of use. Further, in some embodiments, the liquid-reservoir system 8 administers at least about 50% of the (s)-ketorolac in the reservoir system during approximately 24 hours of use.

As shown in FIG. 2, a method for treating moderate to severe pain transdermally and/or topically includes providing a liquid-reservoir system formed of a backing layer, a liquid-reservoir layer, a membrane layer, a contact adhesive layer, and a release liner layer, as shown at functional block S100. In some embodiments, the liquid-reservoir layer comprises an active drug ingredient, one or more permeation enhancers, an antioxidant, water, and a buffering agent (i.e., pH adjuster). In some embodiments, the active drug ingredient includes, at least, (s)-ketorolac. In some embodiments, the provided liquid-reservoir system includes any one of the system embodiments described above. As shown at functional blocks S110-S130, the pain treatment method further includes: removing the release liner layer from the liquid-reservoir system by peeling away the release liner layer (S110); positioning the liquid-reservoir system onto a portion of a patient's skin with the backing layer facing outward from the portion of the patient's skin (S120); and applying pressure to the backing layer to non-permanently adhere the liquid-reservoir system to the portion of the patient's skin (S130). In some embodiments, the liquid-reservoir system is remains adhered to the patient's skin for an extended period of time. In some embodiments, the liquid-reservoir system remains adhered to the patient's skin for at least 24 hours.

### (S)-Ketorolac Gel Formulation Compositions for Examples

Five (s)-ketorolac gel formulations, as shown in Table 1, were tested in the various examples as described below. The (s)-ketorolac gel formulations were prepared according to the following:

Solution 1: isopropyl alcohol, isopropyl myristate, BHT, and in some embodiments oleic acid, were combined to form a clear solution;

Solution 2: specified amount of(s)-ketorolac was added to Solution 1 and heated at about 35° C. until a clear solution was obtained;

Solution 3: purified water and triethanolamine or epolamine were combined and vortexed for about thirty seconds;

Solution 4: Solutions 2 and 3 were combined and positioned in an orbital shaker and gently shaken for about one hour. The pH of Solution 4 was adjusted to about 5.0 (range 4.5-5.5) using acid (e.g., HCl) or base (e.g., triethanolamine, epolamine). A thickening agent, for example HPC-H, was added to the Solution 4 and immediately dispersed using a hand-held small propeller mixer for about one minute and then placed in an orbital shaker overnight to achieve a uniform clear gel. The final pH of the homogenous gel was measured using a pH meter.

TABLE 1
(s)-Ketorolac gel formulation compositions

| | KRG-1 | KRG-2 | KRG-3 | KRG-4 | KRG-5 |
|---|---|---|---|---|---|
| | (% w/w) | (% w/w) | (% w/w) | (% w/w) | (% w/w) |
| Ingredient | (pH 4.9) | (pH 5.6) | (pH 4.9) | (pH 4.9) | (pH 4.9) |
| (s)-ketorolac acid | 3.2 | 2.9 | 2.9 | 3.0 | 2.9 |
| Isopropyl alcohol (IPA) | 46.9 | 53.9 | 54.1 | 56.6 | 46.8 |
| Isopropyl myristate (IPM) | 1.1 | 1.5 | 1.7 | 2.0 | 1.2 |
| Oleic acid | 0 | 0 | 2.0 | 0 | 0 |
| Butylated hydroxytoluene (BHT) | 0.07 | 0.1 | 0.1 | 0.1 | 0.06 |
| Triethanolamine (TEA) | 1.1 | 0 | 1.4 | 1.1 | 1.1 |
| Epolamine | 0 | 1.5 | 0 | 0 | 0 |
| Purified water | 45.6 | 38.1 | 35.9 | 35.9 | 45.9 |
| Hydroxypropyl cellulose, H (HPC-H) | 2.0 | 2.0 | 2.0 | 1.5 | 2.0 |
| Total weight, gm | 100 | 100 | 100 | 100 | 100 |

### (S)-Ketorolac Liquid-Reservoir System Compositions for Examples

Five liquid-reservoir systems (LRS), as shown in Table 2, were tested in the various examples as described below. A pressure-sensitive adhesive solution was coated on a release side of the release liner layer and immediately dried with a hand-held drier for about one minute followed by air drying for about one hour. The dried adhesive coating weight of the adhesive film was determined to be about 3-4 mg/cm². The dried film was then laminated on to a heat-sealable microporous membrane (e.g., Solupor™) to form a controlled membrane laminate (CML) film. The rectangular shape pouch was fabricated by placing an impermeable heat-sealable backing film on the top of microporous membrane (Solupor) or EVA of CML and heat-sealed three sides and leaving the fourth side open for liquid gel insertion. A known amount of liquid gel was withdrawn in a syringe and dispensed inside the rectangular pouch and immediately heat-sealed the opening to form a LRS. The LRS was then placed in a polyester pouch and then the pouch was also heat-sealed. The gel in the LRS was equilibrated for at least two days before skin flux experiments and other characterizations of LRS patches were performed.

TABLE 2
(s)-Ketorolac Liquid-Reservoir System Compositions

| LRS ID | Liquid gel | Membrane | Contact Adhesive | Heat-sealable backing | Release Liner |
|---|---|---|---|---|---|
| LRS-1 | KRG-5 | 19% EVA | Duro-Tak 2516 | Polyethylene/polyester | Polyester film |
| LRS-2 | KRG-5 | Solupor-10P05A | Duro-Tak 2516 | Polyethylene/polyester | Polyester film |
| LRS-3 | KRG-5 | Solupor-10P05A | Duro-Tak 900A | Polyethylene/polyester | Polyester film |
| LRS-4 | KRG-5 | Solupor-10P05A | Duro-Tak 9301 | Polyethylene/polyester | Polyester film |
| LRS-5 | KRG-5 | Solupor-10P05A | Duro-Tak 4098 | Polyethylene/polyester | Polyester film |

### Example 1

In vitro skin permeation studies of(s)-ketorolac gel formulations were performed using a Franz cell diffusion system, as shown in FIG. 3. A Franz cell diffusion system includes two chambers: a donor chamber and a receiver chamber with a diffusion area of 0.79 cm². About 3 cm² of cadaver skin (Science Care, Phoenix, Ariz.) was die-cut out and a LRS patch (removed release liner before application) of the same size was applied onto a stratum corneum side of the skin with gentle pressure to assure sufficient contact between the skin and the patch. The skin was then positioned on the receiver chamber with the patch side facing the donor chamber and an O-ring was placed on the top of the receiver chamber. The donor chamber was then positioned on the receiver chamber and tightly clamped. In case of baseline gel skin permeation experiments, a known amount of the gel was applied onto the donor chamber in contact with the stratum corneum of the skin. The receiver chamber of the Franz cell diffusion system was filled with phosphate buffered saline (PBS) containing sodium azide (pH 7.4) and a small magnetic stirring bar was placed in the receiver chamber. The assembled Franz cell diffusion system, as shown in FIG. 3, was then positioned on a hot magnetic stirring plate with mixing speed of about 200 rpm and the receiver fluid temperature was maintained at 32° C. At a predetermined time, all of the fluid was emptied from the receiver chamber and then refilled with fresh PBS. The samples were taken at the following intervals: zero hours (to establish the absence of (s)-ketorolac), and then four hours, eight hours, sixteen hours, and twenty-four hours. The skin samples were assayed for (s)-ketorolac using high performance liquid chromatography (HPLC) with ultraviolet (UV) light detection. At least three diffusion cells were used for each gel or patch formulation tested. The cumulative amount of (s)-ketorolac that permeated through the cadaver skin as a function of time was plotted. The skin flux (μg/cm²/h) was determined from the linear slope of the plot of a cumulative amount (μg/cm²) permeated through skin versus time. The lag time was determined from the x-axis intercept of the linear slope.

The mean skin flux of (s)-ketorolac from the five liquid-gel formulations is summarized in Table 3. The values represented in Table 3 are the result of an n=3 experiments. The formulations, KRG-1 and KRG-5 exhibited similar skin fluxes as the formulation compositions were very similar. Although the KRG-2 and KRG-3 formulations were relatively similar except for buffering agent, oleic acid, and difference in gel pH values (5.6 vs. 4.9), the skin flux was roughly 50% lower for KRG-2 than that of KRG-3. This is likely because of high ionization of(s)-ketorolac at higher pHs (e.g., pH 5.6 of KRG-2) leading to low permeation of ionic species as opposed to uncharged and lipophilic species at lower pHs (e.g., pH 4.9 of KRG-3). The KRG-4 skin flux was lower than KRG-3 likely because of a higher percent weight by weight isopropyl alcohol content in KRG-4. Due to the high degree of permeation of KRG-5, KRG-5 was selected for further experiments, as shown in Table 4 and Examples 2-3.

TABLE 3
(s)-Ketorolac Mean Skin Flux Through Cadaver
Skin From Gel Formulations

| | Mean | |
|---|---|---|
| | skin | Lag |
| Liquid gel | Flux | time |
| formulation ID | (μg/cm²/h) | (h) |
| KRG-1 | 45 | 5 |
| KRG-2 | 8 | 5 |
| KRG-3 | 18 | 2 |
| KRG-4 | 7 | 2 |
| KRG-5 | 56 | 5 |

The mean skin flux of (s)-ketorolac liquid-reservoir system compositions is summarized in Table 4. The results represented in Table 4 are a result of an n=3 experiments. The standard deviation of the mean values were less than 20%. The mean skin flux from LRS-1 was several folds lower than those of LRS-2 and LRS-3, suggesting that incorporation of polyethylene into the membrane layer promotes or at least does not inhibit permeation of(s)-ketorolac through cadaver skin as compared to EVA. A bar graph, as shown in FIG. 4, compares mean skin flux from KRG-1, LRS-1, LRS-2, and LRS-3. LRS-2 and LRS-3 exhibited very similar mean skin flux as these patches only differed by contact adhesive layer composition in contact with skin.

TABLE 4
(s)-Ketorolac Mean Flux Through Cadaver
Skin from Liquid-Reservoir System

| | Mean |
|---|---|
| | Skin |
| | Flux |
| LRS formulation ID | (μg/cm²/h) |
| LRS-1 | 0.9 |
| LRS-2 | 17 |
| LRS-3 | 16 |

### Example 2

The stability of the (s)-Ketorolac gel (KRG-5) was determined at 25° C. and 40° C. using two separate stability chambers. The stability sample was withdrawn at the following time points: zero, one, two, three, and seven months. Briefly, an accurately weighed amount of sample was placed in a scintillation vial containing an appropriate amount of undiluted methanol, vortexed for about thirty seconds, and shaken in an orbital shaker for about two hours prior to injecting into an HPLC for drug and related substance (i.e., RS, impurities) assays.

FIG. 5 shows the stability of KRG-5 gel at 25° C. and 40° C. for seven months. No significant changes in (s)-ketorolac percent label strength were observed at 25° C. and 40° C. during a seven month period.

The percent area (Area %) of (s)-ketorolac (KT) and related substance (RS) are summarized in Tables 5-6. As shown in both the gel formulation (FIG. 5) and the liquid-reservoir system (FIG. 6), at 25° C. storage condition, very little RS was observed, while at 40° C. a slight increase in RS with time was observed. The RS is hypothesized to be the (s)-ketorolac isopropyl ester that was formed due to esterification of (s)-ketorolac free acid with isopropyl alcohol.

TABLE 5
Impurity Profiles of (s)-Ketorolac from Gel Formulations

| | KRG-5 | KRG-5 | KRG-5 | KRG-5 |
|---|---|---|---|---|
| | (25° C.) | (25° C.) | (40° C.) | (40° C.) |
| Month | KT, Area % | RS, Area % | KT, Area % | RS, Area % |
| 0 | 100 | 0.0 | — | — |
| 1 | 99.8 | 0.2 | 99.4 | 0.6 |
| 2 | 99.6 | 0.4 | 99.3 | 0.7 |
| 3 | 99.6 | 0.4 | 99.0 | 1.0 |
| 7 | 99.4 | 0.6 | 98.1 | 1.9 |

TABLE 6
Impurity Profiles of (s)-Ketorolac from Liquid-Reservoir System

| | LRS-3 | LRS-3 | LRS-3 | LRS-3 |
|---|---|---|---|---|
| | (25° C.) | (25° C.) | (40° C.) | (40° C.) |
| Month | KT, Area % | RS, Area % | KT, Area % | RS, Area % |
| 0 | 100 | 0.0 | — | — |
| 1 | 99.9 | 0.1 | 99.4 | 0.6 |
| 2 | 99.7 | 0.3 | 99.2 | 0.8 |
| 3 | 99.7 | 0.3 | 99.1 | 0.9 |
| 7 | 99.2 | 0.8 | 97.7 | 2.3 |

### Example 3

As shown in Table 7, skin irritation studies were performed using a 2.9% (s)-ketorolac (KRG-5 in Table 1) LRS (LRS-3 in Table 2) (test or active group), 0% (s)-ketorolac LRS (placebo or negative control group), and a 5% sodium dodecyl sulfate (SDS) (positive control group). New Zealand rabbits were used for skin irritation studies. Five rabbits were used and each rabbit received a placebo LRS, an active LRS, and a positive control (5% SDS). Briefly, the rabbit hairs were carefully shaved using a trimmer prior to patch application. The skin surface was cleaned using rubbing alcohol and dried. The three test LRS were applied separately to each of the five rabbits and wrapped immediately with medical tape. After twenty-four hours, the tape and patches were removed and application sites were scored using standard visual score analogs (VAS) for erythema and edema. The following VAS scale was used for skin irritation (both erythema and edema) scoring: 0: none; 1: slight; 2: mild; 3: moderate; and 4: severe. An average score was determined with n=5 as shown in Table 7. There was no significant irritation observed for patches comprising 3% (s)-ketorolac. Furthermore, there was no significant difference in irritation scores between placebo and active patches suggesting (s)-ketorolac is a non-irritant. On the other hand, the SDS, 5%, as a positive control, exhibited slight-to-moderate skin irritation (erythema and edema), indicating that experimental parameters were set-up appropriately.

TABLE 7
Skin Irritation Scores for Active and Placebo
(s)-Ketorolac Liquid Reservoir System

| | Overall | Overall |
|---|---|---|
| | Erythema | Edema |
| Test/Control Article | Score | Score |
| (s)-Ketorolac LRS patch, 3% | 0.00 | 0.00 |
| Placebo LRS patch | 0.00 | 0.00 |
| SDS, 5% | 1.0 | 2.0 |
| (positive control) | | |

Unless otherwise defined, each technical or scientific term used herein has the same meaning as commonly understood by one of ordinary skill in the art to which this disclosure belongs.

As used in the description and claims, the singular form “a”, “an” and “the” include both singular and plural references unless the context clearly dictates otherwise. For example, the term “reservoir layer” may include, and is contemplated to include, a plurality of reservoir layers. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or weight percent), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the system, composition, formulation, or method includes the recited elements, and may additionally include any other elements. “Consisting essentially of” shall mean that the system, composition, formulation, or method includes the recited elements and excludes other elements of essential significance to the combination for the stated purpose. Thus, a formulation consisting essentially of the elements as defined herein would not exclude other compounds or substances that do not materially affect the basic and novel characteristic(s) of the claimed invention. “Consisting of” shall mean that the system, composition, formulation, or method includes the recited elements and excludes anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1-30. (canceled)

31. A liquid-reservoir system for transdermal administration of (s)-ketorolac, the system comprising:
a backing layer that is substantially impermeable to (s)-ketorolac, isopropyl alcohol, isopropyl myristate, oleic acid, butylated hydroxytoluene, triethanolamine, hydroxypropyl cellulose, and water, wherein the backing layer defines a face surface of the liquid-reservoir system; and
an (s)-ketorolac reservoir layer comprising a liquid-reservoir gel formulation, wherein the liquid-reservoir gel formulation comprises:
1-15% by weight (s)-ketorolac;
one or more drug solubilizing vehicles;
one or more permeation enhancers;
an antioxidant;
a thickening agent;
a purified water; and
a buffering agent.

32. The liquid-reservoir system of claim 31, wherein the liquid-reservoir system exhibits in vitro skin flux through a skin surface of a cadaver in a range of 5 to 100 μg/cm²/hr.

33. The liquid-reservoir system of claim 31, wherein the one or more drug solubilizing vehicles is selected from the group consisting of: isopropyl alcohol and ethanol.

34. The liquid-reservoir system of claim 33, wherein the one or more drug solubilizing vehicles form 30% to 80% of the liquid-reservoir gel formulation by weight.

35. The liquid-reservoir system of claim 31, wherein the purified water forms 20% to 70% of the liquid-reservoir gel formulation by weight.

36. The liquid-reservoir system of claim 31, wherein the backing layer is occlusive and heat-sealable to a membrane layer.

37. The liquid-reservoir system of claim 31, wherein a basal surface area of the liquid-reservoir system is 10 to 100 cm².

38. The liquid-reservoir system of claim 31, further comprising a membrane layer comprising an ethylene-vinyl-acetate membrane with an amount of vinyl acetate between 9% and 28%.

39. The liquid-reservoir system of claim 31, wherein the one or more permeation enhancers is selected from the group consisting of: isopropyl myristate, oleic acid, propylene glycol monolaurate, and a combination thereof.

40. The liquid-reservoir system of claim 31, wherein the antioxidant is selected from the group consisting of: tocopherol, butylated hydroxytoluene, and a combination thereof.

41. The liquid-reservoir system of claim 40, wherein one or more of the tocopherol and butylated hydroxytoluene form 0.5% to 3% of the liquid-reservoir gel formulation by weight.

42. The liquid-reservoir system of claim 31, wherein the thickening agent is selected from the group consisting of: hydroxypropyl cellulose, carboxymethyl cellulose, hydroxyethyl cellulose, and a combination thereof.

43. The liquid-reservoir system of claim 31, further comprising a membrane layer comprising a microporous polyethylene membrane or an ethylene-vinyl-acetate membrane containing vinyl acetate between 4% and 28%.

44. The liquid-reservoir system of claim 43, wherein a thickness of the membrane layer is 0.02-0.2 mm.

45. The liquid-reservoir system of claim 43, wherein one or more of the isopropyl myristate, oleic acid, and propylene glycol monolaurate form 0.5% to 10% of the liquid-reservoir gel formulation by weight.

46. The liquid-reservoir system of claim 43, wherein one or more of the hydroxypropyl cellulose, carboxymethyl cellulose, and hydroxyethyl cellulose form 0.5% to 5% of the liquid-reservoir gel formulation by weight.

47. The liquid-reservoir system of claim 31, further comprising a continuous or peripheral contact adhesive layer comprising:
an adhesive selected from the group consisting of: an acrylates copolymer, a silicone, a polyisobutylene pressure-sensitive adhesive, and a combination thereof; and a tackifier selected from the group consisting of: a mineral oil and a silicone oil.

48. The liquid-reservoir system of claim 47, wherein one or more of the mineral oil and the silicone oil forms 1% to 5% of the contact adhesive layer by weight.

49. A method of transdermal drug delivery, the method comprising:
providing a liquid-reservoir system comprising a backing layer, a liquid-reservoir layer, a membrane layer, a contact adhesive layer, and a release liner layer, wherein the liquid-reservoir layer comprises an active drug ingredient, one or more drug solubilizing vehicles, one or more permeation enhancers, an antioxidant, purified water, and a buffering agent;
removing the release liner layer from the liquid-reservoir system by peeling away the release liner layer;
positioning the liquid-reservoir system onto a portion of a patient's skin with the backing layer facing outward from the portion of the patient's skin; and
applying pressure to the backing layer to non-permanently adhere the liquid-reservoir system to the portion of the patient's skin.

50. The method of claim 49, wherein the active drug ingredient is (s)-ketorolac or a salt thereof.
